(19) United States
(12) Patent Application Publication
(10) Pub. No.: US 2018/0008836 A1
(43) Pub. Date: Jan. 11, 2018

(54) PHOTON ENHANCED BIOLOGICAL SCAFFOLDING

(71) Applicant: JONATHAN K. GEORGE, Arlington, VA (US)

(72) Inventor: JONATHAN K. GEORGE, Arlington, VA (US)

(21) Appl. No.: 15/544,154

(22) PCT Filed: Feb. 18, 2016
(86) PCT No.: PCT/US16/18534

§ 371 Date: Jul. 17, 2017

Related U.S. Application Data
(60) Provisional application No. 62/117,515, filed on Feb. 18, 2015.

(51) Int. Cl. A61N 5/06; A61L 27/50; G02B 6/12; C12M 3/00; C12M 1/12

(52) U.S. Cl. CPC A61N 5/0601; C12M 21/08; C12M 25/14; G02B 6/12002; A61L 27/507; A61L 2430/02; A61L 2430/30; A61L 2430/32; A61N 2005/063

(57) ABSTRACT

Provided herein are biocompatible scaffolds engineered to convey growth stimulatory light to cells and augment their growth on the scaffolds both in vitro and in vivo. Also provide are methods of modifying biocompatible transparent waveguides to control delivery of light from the waveguide material.

### CROSS REFERENCE TO RELATED APPLICATIONS

This application claims priority based on U.S. Provisional Application Ser. No. 62/117,515 filed Feb. 18, 2015, which is incorporated herein by reference.

### FIELD OF THE INVENTION

This invention relates generally to compositions and methods for growth of cells on scaffolds both in vivo and in vitro.

### BACKGROUND OF THE INVENTION

Without limiting the scope of the invention, its background is described in connection with existing repair of damaged tissue. Treatment of disease or injury has historically focused on transplanting tissue from one site to another in the same patient (autografts) or from one individual to another (transplants or allografts). Problematically, the harvesting of autografts is expensive, painful, has anatomical constraints and results in injury to the donor site. Allografts are also highly problematic with constraints on availability, the potential for rejection and the risk of disease transmission. As an alternative, tissue engineering (a.k.a. regenerative medicine) hopes to provide augmented regeneration of damaged tissues, in lieu of replacement. In particular, tissue engineering (TE) hopes to augment the repair of damaged tissues by combining cells with porous scaffold biomaterials that act as templates for tissue regeneration and that enhance whatever natural repair and regeneration process might otherwise occur. Key historical requirements for an acceptable TE scaffold include biocompatibility, biodegradability, adequate mechanical properties depending on the indication, and a scaffold architecture that provides high interconnectivity and porosity to allow cellular penetration and remodeling and diffusion of nutrients to cells within the construct as well as waste products away from the cells.

Another desirable aspect to an ideal tissue scaffold would be the ability of the scaffold to provide regenerative signals to enhance the speed and integrity of cell growth on the scaffold. This would allow the more rapid generation of autologous scaffolds in vitro and thus a shortening time between cell seeding and implantation but may ideally negate the need for in vitro culture prior to implantation. To this end, current research is being directed enhancing cell behavior through delivery of biological and biochemical signals including adapting the scaffold as a delivery system for growth factors, adhesion peptides and cytokines. However the addition of biological and biochemical signals to a scaffold promises a more prolonged regulatory process to enter clinical availability.

From the foregoing, it appeared to the present inventor that the ability to augment cell growth on biodegradable scaffolds would be particularly desirable and would answer a long felt need in the industry. Provided herein is the discovery of novel compositions, apparatus and methods for augmenting the growth of cells on biocompatible scaffolds using photobiomodulation.

### BRIEF SUMMARY OF THE INVENTION

Provided herein are compositions and methods that apply a photobiomodulation dosage of stimulating light to subsurface wounds by controlling light scattered through embeddable biodegradable optical fibers and waveguides. Transparent optical waveguides are formed as biological scaffolds and implants to distribute light evenly throughout the tissue to reach optimal application of photobiomodulation.

In one embodiment a device for tissue repair is provided that includes a tissue scaffold formed of a plurality of interconnected photon waveguides, the waveguides adapted convey cell stimulatory photons and to release the cell stimulatory photons from the waveguides by optical scattering, and an optical connector attached to the tissue scaffold, wherein the optical connector is adapted to connect to a source of cell stimulatory photons. The waveguides of the device are biodegradable in certain embodiments and non-limiting examples of such waveguides include transparent polylactide (PLA), silk fibroin, and polyethylene glycol (PEG).

The waveguides are adapted for controlled optical scattering. In one embodiment the optical scattering is controlled by forming the tissue scaffold is formed as a plurality of interconnecting ring resonators, which can be formed as an essentially 2 dimensional (2D) sheet or as a three dimensional (3D) mesh like structure. Thus, in certain embodiments a three-dimensional biocompatible tissue scaffold is provided that includes a biocompatible transparent material that conducts photons provided from a photon source and releases the photons substantially evenly from the transparent material forming the scaffold, wherein the scaffold is formed as an interconnecting array of ring resonators that includes a plurality of interconnected voids dimensioned to allow movement of cells having an average diameter of 10-30 μm (microns) through the scaffold.

In certain embodiments, the waveguides are composed of PLA or silk fibroin and are treated by surface etching to increase optical scattering while in other embodiments the waveguides are heat treated to generate amorphous boundary layers that result in increased optical scattering.

The tissue scaffolds provided herein may be employed in a number of medical indications and can thus be formed as expandable stents, or as bone, muscle, vascular or nervous tissue repair scaffolds. In one embodiment the tissue scaffold is a 3D printed anatomically correct ear or nose prosthesis. In other embodiments, the tissue scaffold is dimensional and adapted as a hernia repair scaffold.

In certain embodiments, the tissue scaffold is connected to a light source through a dual use connector that includes a central fluid conduit that provides fluid flow into and away from the tissue scaffold.

In particular embodiments the optical conduit that connects to the tissue scaffold is adapted to connect to a laser or light emitting diode as a source of cell stimulatory photons elaborated by a laser or light emitting diode. In certain embodiments the laser or light emitting diode that emits cell stimulatory photons in one or more wavelengths in a range of wavelengths from 620 nm to 760 nm.

Also provided are methods of making a cell seeded tissue scaffold that includes providing a tissue scaffold into a sterile in vitro cell growth chamber, wherein the tissue scaffold comprises a plurality of interconnected photon waveguides, the waveguides adapted convey cell stimulatory photons and to release the cell stimulatory photons from the waveguides by optical scattering. The tissue scaffold is connected to a source of cell stimulatory photons, is seeded with a plurality of cells in a growth medium; and incubated under conditions and for a time sufficient for the cells to colonize the scaffold.

### BRIEF DESCRIPTION OF THE DRAWINGS

For a more complete understanding of the present invention, including features and advantages, reference is now made to the detailed description of the invention along with the accompanying figures:

FIG. 1A is a photograph of a test apparatus built to apply light to a waveguide and to measure scattering. FIG. 1B is a photograph of an optically modified PLA waveguide transmitting red laser light and showing markedly increased optical scattering visible to the naked eye. FIG. 1C is a photograph of an optically modified PLA waveguide transmitting red laser light in a dark box and showing markedly increased optical scattering visible to the naked eye.

FIG. 2 provides an Atomic Force Microscope (AFM) image of the surface of a PLA fiber prior to treatment.

FIG. 3 provides a photographic image of the effect of increasing concentration of NaOH on light scattering from the surface of a light wave compared with the untreated PLA waveguide.

FIG. 4A provides an AFM image of the surface of a PLA fiber treated with 0.5 M NaOH. FIG. 4B is a slice of AFM showing increased roughness (range in Y axis) vs FIG. 2.

FIG. 5A provides an AFM image of the surface of a PLA fiber treated by microwave heat in the presence of surface water. FIG. 5B is a slice of AFM showing increased roughness (range in Y axis) vs FIG. 2.

FIG. 6A provide photographic images of the red light emanating from the surface of a PLA waveguide with increased microwave time. As shown in FIG. 6B, with each 10 second increment of the microwave interval the scattering first increased and then over time absorption dominated as shown in FIG. 6A and FIG. 6B.

FIGS. 7A-D provide further analysis of the effects of microwave time and scattering. FIG. 7A shows that the combination of scattering and absorption increase with microwave exposure time. FIG. 7B shows scattering increasing with microwave time. FIG. 7C shows absorption over microwave time. The final plot in FIG. 7D shows optical amplitude.

FIG. 8 provides a cartoon of cells growing on a photon transparent scaffold illuminated by photons from a light source.

FIG. 9 provides a 3D rendering of cells adhered to a photon transparent scaffold.

FIG. 10A graphically depicts the movement of light through a ring resonator. FIG. 10B depicts a row in linked ring resonators while FIG. 10C depicts a 2D array of ring resonators including rings that have a width of the waveguide relative to the radius of the other rings such that more light will be released by thicker rings. FIG. 10D provides another embodiment of a ring resonator array that permits the spread of light evenly over the entire array.

FIG. 11A graphically depicts a side view of a photon transparent scaffolding having a light coupling conduit emanating generally orthogonally to the plane of the scaffold. FIG. 11B depicts a close-up view of a scaffold having a catheter attachment tube.

FIG. 12 depicts an underside view of the scaffold of FIG. 11B and shows a bottom opening of the catheter-coupling tube that allows liquid to flow into and out of the catheter.

FIG. 13 depicts a three dimensional embodiment of a mesh scaffold implant formed by a plurality of ring resonators.

FIG. 14A provides a side schematic of one embodiment wherein each set of vertical rings couples sets of 2D horizontal ring arrays to form a three dimensional structure. In the front schematic of FIG. 14B, curved vertical rings connect layers of curved horizontal rings.

FIG. 15A provides a cross section of one embodiment of a dual use light conduit catheter combination. FIG. 15B depicts an embodiment including a connecting flange around an end of dual use light conduit catheter combination that provides a mechanical connection to the scaffold by applied radial tension. FIG. 15C shows an end view of dual use conduit within the flange.

FIG. 16A provides an embodiment of a splitter for a dual use light conduit catheter combination where the central conduit leaves the combined light conduit. In this embodiment a small angle joint is provided to prevent optical scattering from the optical path as it enters the combined light conduit catheter. FIG. 16B depicts one embodiment of a capillary wave guide.

FIG. 17A depicts placement of a photobiomodulation scaffold in a patient. In the embodiment depicted in FIG. 17B, a dual use light conduit catheter is provided. In alternative embodiments such as depicted in FIG. 17C, the light source may be inside the body and optically coupled to the scaffold or may be located on or in the scaffold as depicted in FIG. 17D.

FIG. 18A depicts a light conductive photobiomodulation scaffold placed in a bioreactor. The light source can be external to the bioreactor as in FIG. 18A or can be located within the bioreactor as depicted in FIG. 18B. FIG. 18C depicts an optical splitter placed in the optical path between the light source and the scaffold to collect light reflecting back from the scaffold such that the reflected light can be sent to an optical sensor.

FIG. 19A depicts the decay in light scattering over distance through a transparent waveguide. FIG. 19B depicts a mirror positioned at the end of the waveguide to reflect back the power emanating from laser. The effect of this is shown figuratively in FIG. 19C where the vertical line shows placement of the mirror and the effect of mirror placement on ameliorating scattering decay is shown.

FIG. 20A depicts a photobiomodulation scaffold utilized in repair of a joint. FIG. 20B depicts a photobiomodulation scaffold utilized in repair of a bone defect. FIG. 20C depicts photobiomodulation scaffold adapted and dimensioned for use as a stent. FIG. 20D depicts a partial side view of a photobiomodulation scaffold adapted to provide tracts for guiding nerve growth. FIG. 20E provides an end on view of the embodiment of FIG. 20D.

### DETAILED DESCRIPTION OF THE INVENTION

Provided herein are biocompatible scaffolds that are engineered to convey growth stimulatory light to cells and augment their growth on the scaffolds both in vitro and in vivo. Photobiomodulation (PBM), also known as Low-Level Light Therapy (LLLT), is the application of light to living cells to increase, decrease, or otherwise modulate biomolecules within the cells. Photobiomodulation applies of light of specific frequencies to cells to stimulate tissue generation, guide cell growth, reduce inflammation, and otherwise modulate biological activity. Useful wavelengths are in the visible to near-infra-red spectrum from 380 to 760 nm. In particular embodiments a range of wavelengths from about 620 nm to about 760 nm is employed. A primary target of PBM is mitochondria. By increasing mitochondrial activity the cellular metabolism is increased, this increases the amount of Adenosine Triphosphate (ATP) available to the cell and in turn enhances cell viability and increases cell production in tissue.

Electron transport in eukaryotes is via the oxidative phosphorylation metabolic pathway whereby nutrients are oxidized to form ATP. The process takes place on the inner membrane of mitochondria and proceeds through a series of enzymatic processes beginning with NADH dehydrogenase (Complex I) through succinate dehydrogenase (Complex II) via the citric acid cycle. The process continues through the action of ubiquinone cytochrome C oxidoreductase (to Complex III) and concludes with cytochrome C oxidase (Complex IV). The hypothesized pathway for the photochemical effect is the photon induced disassociation of the binding of nitric oxide to iron and copper redox centers in cytochrome c oxidase of the mitochondria. Cytochrome c oxidase is the fourth and final enzyme of the mitochondrial electron transport chain controlling cellular metabolism.

PBM was discovered more than 50 years ago, but has not been widely adopted in the clinical setting and when utilized is primarily limited to external sources. Under existing PBM protocols, light is directed from outside of the patient towards the skin, muscle, or other diseased tissues. Externally sourced photobiomodulation is limited by the exponential optical absorption of most biological tissue. While damaged tissue has been shown to regenerate faster when illuminated with red to near-infrared light, tissue strongly absorbs light within this frequency range, limiting the clinical use of photobiomodulation to surface wounds and to in vitro tissue incubators. Due to the strong optical absorption, it is impossible to dose deep tissue with external photobiomodulating light without overdosing the tissue near the surface. The photobiomodulation dose, like many pharmacological agents, follows a biphasic response. A low dose results in a marginal benefit, an optimal dose results in the greatest benefit, and an overdose has a detrimental effect. With a biphasic response, externally sourced photobiomodulation will never reach optimal dose across the entire depth of the tissue.

The present inventor appreciated that clinical application of photobiomodulation would require the development of controlled intratissue biologically compatible delivery mechanisms and so developed the novel materials, structures and methods described herein. Thus, provided herein are compositions and methods that apply a photobiomodulation dosage of light to subsurface wounds by controlling light scattered through embeddable biodegradable optical fibers and waveguides. The transparent optical waveguides are formed as biological scaffolds and implants to distribute light evenly throughout the tissue to reach optimal application of photobiomodulation.

Optical Delivery:

There are several options for delivering light into a photobiomodulation scaffold. The simplest option is to direct a beam of light onto the surface of the scaffold. The areas of the scaffold that are exposed to the surface of the body or tissue will collect light from the beam and direct it into the tissue. This option is most suited to tissue growing in vitro in a bioreactor where it is possible to directly expose the scaffold to the light beam. A second option is to deliver the light with a biomedical fiber optic. In this option light is directed from an LED or laser source outside of the body or growing tissue through a fiber optic cable connected into the optical scaffold. A third option is to use a specially designed catheter with tube made from a transparent material to act as a waveguide. This allows liquid to be moved into and out of the scaffold as well as directing light into the scaffold. A further option is to source the light from within the scaffold. In this method the light source, either an LED, laser, or luminescent polymer, is embedded in the scaffold. The light source can be powered by an embedded battery or externally by induction.

Tissue Waveguides:

Several categories of waveguide can be used to diffuse light throughout tissue. These include strand waveguides, mesh waveguides, and capillary waveguides. Suggested types of waveguides for different applications as shown in Table 1 below:

TABLE 1
Waveguide Types

| Waveguide | Examples of Manufacturing | | |
|---|---|---|---|
| Type | Processes | Applications | Resonance |
| Strand | Extrusion, laser cutting, injection | Hip replacement, | Fabry-Pérot |
| | molding, stereo lithography, 3D | spinal cord implants | |
| | printing | | |
| Mesh | Laser cutting, injection molding, | Bone implants, brain | Ring |
| | stereo lithography, 3D printing | implants, stents, | |
| | | food protein | |
| Capillary | Injection molding, stereo | Organ tissue, food | None |
| | lithography, 3D printing | protein | |

Strand waveguides are the simplest. These are strands of rectangular or circular transparent material laid through the tissue scaffold. They can be manufactured through extrusion, laser cutting, injection molding, stereo lithography, or 3D printing. They may consist of a single material or a cladding with low refractive index relative to a core with higher refractive index as in a fiber optic. The material must be biocompatible, at least partially transparent, and optionally biodegradable. Materials that meet these requirements include Polylactic Acid (PLA), silk fibroin, and Polyethylene Glycol (PEG) hydrogel. The strands may be manufactured by extrusion, laser cutting planar sheets of the material, injection molding, stereo lithography, or extrusion 3D printing. The transparent material has a higher index of refraction than the primarily water heavy tissue surrounding it and will act as an optical waveguide to light fed into it. Each strand of the waveguide is connected to an optical source, either outside of the tissue, or embedded within the tissue.

Another tissue waveguide type is formed as a mesh. The mesh waveguide consists of an array of rings. Each ring is connected to its neighbor either directly or through a lower refractive index cladding material. The cladding material, as in the strand waveguide, acts like the cladding in fiber optics to contain light within the rings. Together the mesh allows light to be distributed throughout the scaffold without chemically isolating the growing tissue growing around and within the mesh. The mesh waveguide has the additional benefit of each ring acting as an optical resonator. The optical resonance allows light to be distributed evenly throughout the scaffold and is discussed further in the resonance section below.

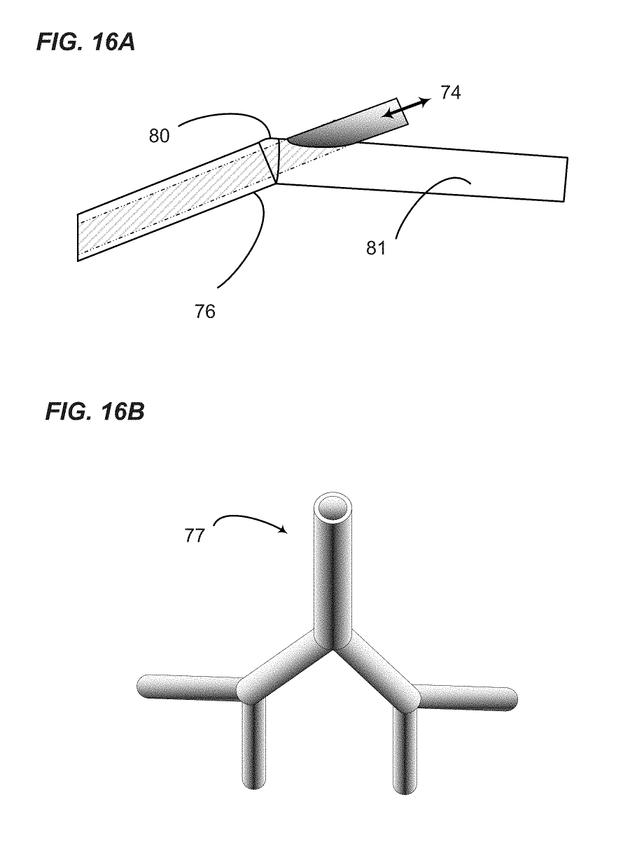

The most complex waveguide type is the capillary waveguide. As is depicted in FIG. 16B, the capillary waveguide (77) is made from a branching structure of transparent tubing. The inner portion of the tube acts as a liquid delivery structure for moving nutrient-rich growth media or blood throughout the growing tissue while the transparent wall of the tube acts as an optical waveguide distributing photobiomodulating light throughout the growing tissue. The 3D nature of the capillary structure requires that this scaffold type be manufactured through injection molding, stereo lithography, or 3D extrusion printing. The benefit of this structure is that the same capillaries required for delivering nutrients to the deep tissue are able to deliver light to the deep tissue. This waveguide type is most suited to scaffolds for organ growth.

Optical Fiber Materials:

Where optical fibers are utilized to direct and supply light to cells, suitable materials for forming the optical fibers must meet the key requirements criteria of biocompatibility, biodegradability, adequate mechanical properties depending on the indication, and ability to be formed with a scaffold architecture that provides high interconnectivity and porosity to allow passage of cells and fluid flow through the construct. However, in order to permit photomodulation the material must also be able to conduct photons.

Polymeric materials are generally transparent when either fully crystalline or fully amorphous. In contrast, when a polymer is inhomogeneous and includes subwavelength regions that mix crystalline and amorphous forms, light is diffused by the boundary condition at the interface between the two different forms of the polymer. Because the light propagation vectors are randomized by the boundaries, any light entering the polymer as a coherent beam will lose its coherence and beam shape as it moves through the diffusing material.

However, in order for the light to reach the tissue it must leave the fiber. As appreciated by the present inventor, light can leave an optical fiber through several theoretical mechanisms including surface defect scattering, subsurface inhomogeneous boundaries, or crystal scattering.

A simple model of the light lost in propagation through an optical fiber or waveguide is given by an ordinary differential equation where the amount of light available to scatter and to be absorbed both decay exponentially with distance. This is shown in Eq. 1 and Eq. 2 where α is the proportional loss due to scattering and β is the proportional loss due to absorption.

$$\begin{matrix}\frac{dP}{dx} = - ( α + β ) P & ( 1 ) \\ P ( x ) = {e}^{- ( α + β ) x} & ( 2 )\end{matrix}$$

To apply an even dose of light to the tissue, the optical waveguide must make Eq. 2 constant by forcing scattering and absorption to vary as functions of 1/x as reflected in Eq. (3) and (4):

$$\begin{matrix}P ( x ) = {e}^{- ( A ( x ) + B ( x ) ) x} & ( 3 ) \\ A ( x ) = \frac{α}{x} , B ( x ) = \frac{β}{x} & ( 4 )\end{matrix}$$

In another example of a mathematical model the optical power in the fiber at a point X is given by Eq. 5:

P(x)=Ae^{x(-α-β)} (5)

Where alpha (α) is the fiber scattering coefficient, beta (β) is the absorption coefficient, and A is the input power.

Power scattered from the surface of the fiber is a function of alpha per Eq. 6:

dPs/dx=αAe^{(x(-α-β))} (6)

Optical power absorbed by the filter is a function of bet per Eq. 7:

dPa/dx=βAe^{(x(-α-β))} (7)

To solve for the three unknowns, alpha (α), beta (β) and A, three measurements are taken:
1: Power at the end of the fiber;
2. Curve fit to the combined alpha+beta from an image; and
3. Power scatter from the surface through a small hole.

$$\begin{matrix}2 ( {P}_{ms} ) = {\int }_{{l}_{1}}^{{l}_{2}} α {Ae}^{( - α - β ) x} dx \\ = \frac{α A ( {e}^{- ( α + β ) {l}_{1}} - {e}^{- ( α + β ) {l}_{2}} )}{α + β}\end{matrix} \qquad ( 8 )$$

The scattering mathematically described above (Eq. 7) can be achieved in practice by modulating the surface defects such as by increasing the etch time, as in the case of an NaOH etching process of biocompatible polymer treatment, disclosed in Example 2 herein, or modulating the subsurface inhomogeneous boundaries such as through modulation of heating temperature, as in the case of the water vapor process, disclosed in Example 3 herein, over the length of the PLA to introduce the 1/x functions in both the proportional scattering and proportional absorption. Several of these mechanisms are exemplified herein using transparent PLA as one non-limiting example of a suitable optical fiber.

Material Processing:

The waveguide material may be modified to increase optical scattering along the length of the strand. Many types of material processing may be employed to increased optical scattering. Several exemplary methods are disclosed herein including boiling in water, application of microwave, high power laser light for surface ablation, or chemical etching with a solvent such as NaOH or acetone. Of the materials and processes exemplified herein, PLA, fibrin and PEG are amenable to microwave or laser ablation as well as chemical etching. PLA may also be modified by boiling to increase optical scattering. The goal of material processing is to design optical scattering out of the waveguide as a function of distance from the optical source. Waveguide material near the optical source is allowed to scatter less light than material at a distance from the source. This flattens the distribution of light over the entire scaffold reducing variations in dose to tissue growing in one section of the scaffold to that of tissue growing in another section allowing the entire scaffold to reach a more optimal dose point.

Structural Modifications:

While modifying scattering of materials through processing can make the scattering a function of distance from the source and more evenly spread the optical power scattered over the surface of the scaffold, this approach may in certain circumstances and scaffold designs be limited by the exponential decay function which may flatten the scattering function to a limited degree. Thus, in a further embodiments the entire scaffold was made into an optical resonator with decreased surface scattering.

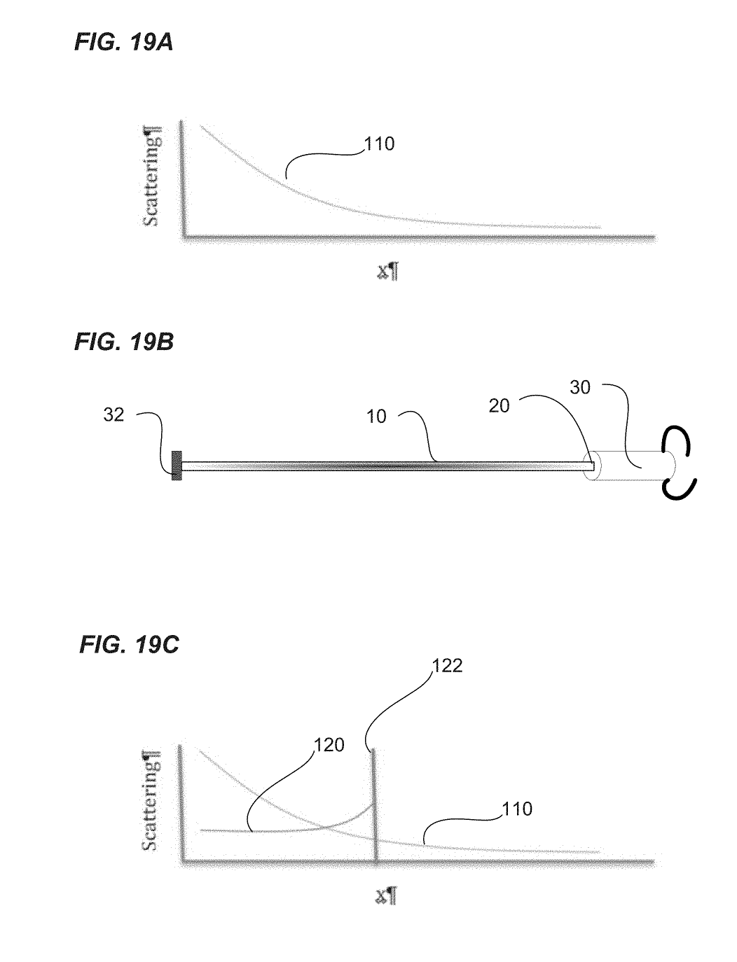

The scattering from optical waveguides like the ones modified by microwaving, boiling, and with NaOH, it was determined as depicted in FIG. 19A that the amount of optical power 110 is greater at first and then drops off with distance according to Eq. 9:

I_{scattered}=αAe^{-αx} (9)

Where α is the power of light scattered

$$\frac{dl}{dx}$$

from the waveguide with length. Solving for a function ƒ_{α}(x) to make I_{scattered} a constant is not possible.

$$\begin{matrix}{I}_{scattered} = {f}_{α} ( x ) {Ae}^{- {f}_{α} ( x ) x} & ( 10 ) \\ ln ( \frac{{I}_{scattered}}{{Af}_{α} ( x )} ) = - {f}_{α} ( x ) x & ( 11 ) \\ ln ( {I}_{scattered} ) = ln ( {Af}_{α} ( x ) ) - {f}_{α} ( x ) x & ( 12 )\end{matrix}$$

Because no solution can entirely flatten exponential scattering according to the above equations, new solutions were employed.

While it is impossible to design an optical scattering material process to flatten the optical intensity over the tissue scaffold, it is possible to design a scaffold with an even intensity profile. By reflecting light back into the waveguide at the waveguide boundary or by directing the light back on to its own path it is possible to entirely level the intensity profile making a perfectly even photobiomodulation dose achievable. When both ends of an optical waveguide are made to be reflective by partially coating with a reflective material such as gold, or creating a significant change in refractive index, the waveguide becomes a Fabry-Perot optical resonator according to Eq. 13:

I_{scattered}(x)=αAe^{-αx}+α(Ae^{-αl})e^{α(l-x)}+ . . . (13)

As the light is reflected back and forth along the resonator the number of terms in the summation becomes greater and the value of I_{scattered} at any point along the waveguide is reduced to a constant.

In one embodiment, as depicted in FIG. 19B, a mirror (32) was positioned at the distal end of the waveguide (10) to reflect back the power emanating from laser (30), which is located at the proximal end of waveguide (10). The effect of this is shown figuratively in FIG. 19C where the vertical line (122) shows placement of the mirror and line (120) shows the effect of mirror placement on ameliorating scattering decay from the light entering the scaffold.

The sum of the line (110) and (120) is now flatter than just line (110) was to begin with. If slightly mirrored caps are placed on both the beginning and the end of the scattering waveguide, such as for example by sputtering the ends with gold to create partially reflective surfaces, the reflections can be made to repeat again and again from both sides. The I_{scattered} function becomes a sum of exponential decay functions. The longer the light is trapped in the resonator the flatter the I_{scattered} function becomes. At the limit the scattering function is completely flat.

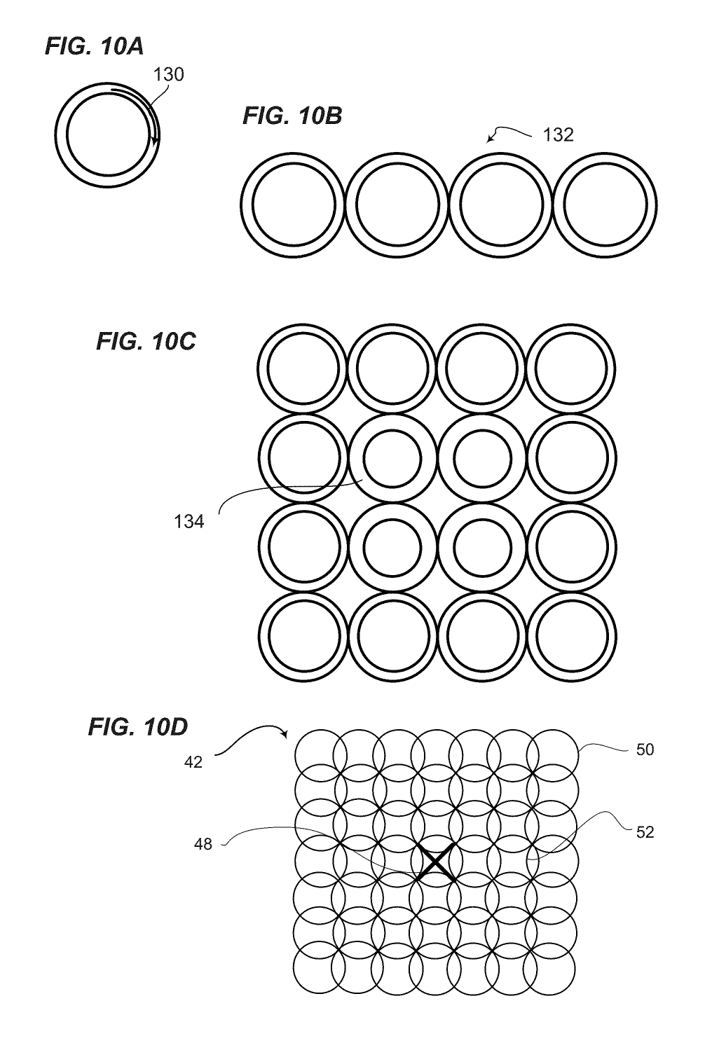

This approach is not restricted to Fabry-Pérot resonators. Ring resonators such as graphically depicted in FIG. 10A are a second resonant optical structure. In a ring resonator the light (130) is made to loop back on itself. FIG. 10B shows are plurality (132) of ring resonators.

Again the I_{scattered} function becomes a sum of exponential decay functions and flattens in the limit. By creating a mesh of these resonators as depicted in FIG. 10D the optical scattering can be spread evenly over the surface of the entire scaffold. (Or any structure.)

Furthermore, the amount of light released by a ring resonator is controlled by the so-called bending losses. If the curve of the ring is made tighter relative to the width of the waveguide, the ring resonator loses more light. This allows us to control the scattering intensity over the scaffold by varying the width of the waveguide relative to the radius of the ring as shown in FIG. 10C, where more light will be released by thicker rings (134).

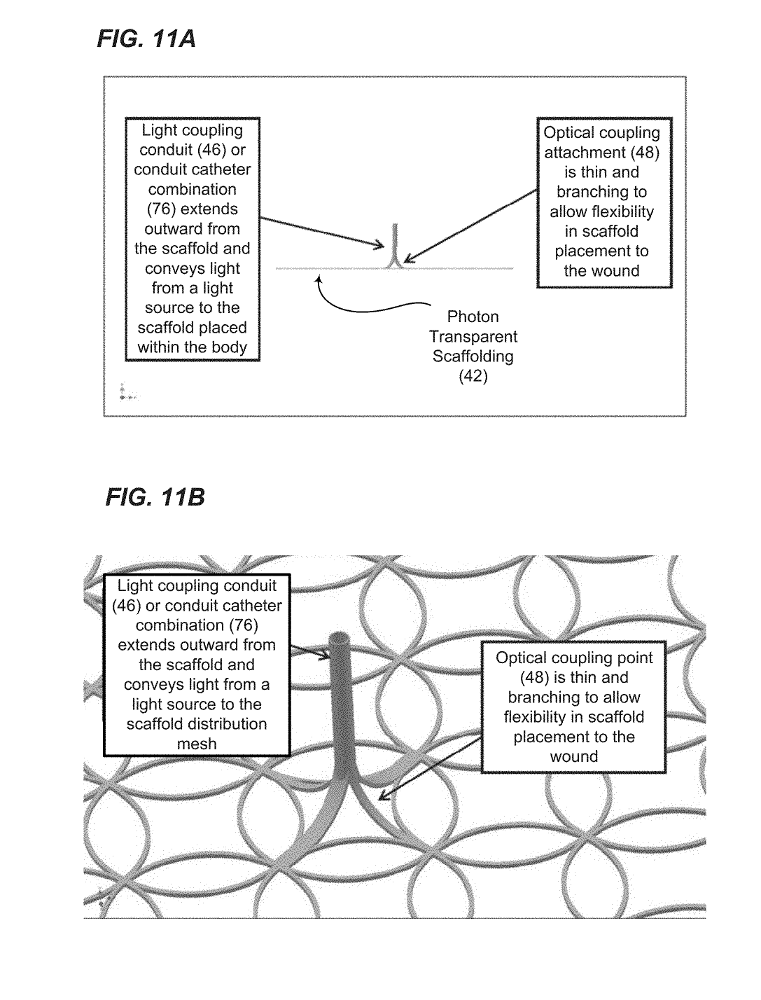

In one embodiment depicted in a top down view of a 2D scaffold implant in FIG. 10D, a mesh like scaffold (42) is provided where the optically transparent regions of the scaffold are created with thin curved surfaces. Curved circular structures become ring resonators (50) and coupling points (52) in the scaffold allow the light to be evenly distributed over the scaffold after entry of the light through optical coupling (48), which will be describe in more detail in reference to FIG. 11B. The thin curved surfaces of the curved circles (50) act with Snell's law to limit the amount of light leaving the scaffold in any given region. Sharp boundaries in the transparent material are avoided, as they would cause regions of high optical scattering in the scaffold. This would expose some regions of tissue to higher optical power than other regions, preventing the optimal power dose from being obtained throughout the tissue. In some embodiments however, scaffold may be designed with points or regions having sharp boundaries that form light delivery zones in the scaffold. The ring resonators can overlap with each other and/or connect at those overlapping sections.

While the making and using of various embodiments of the present invention are discussed in detail below, it should be appreciated that the present invention provides many applicable inventive concepts which can be employed in a wide variety of specific contexts. The specific embodiment discussed herein are merely illustrative of specific ways to make and use the invention and do not delimit the scope of the invention.

The following examples are include for the sake of completeness of disclosure and to illustrate the methods of making the compositions and composites of the present invention as well as to present certain characteristics of the compositions. In no way are these examples intended to limit the scope or teaching of this disclosure.

### Example 1: Engineering to Optically Balance Light Delivery Through a Filament

Polylactic acid or polylactide (PLA) is a biodegradable thermoplastic aliphatic polyester typically derived from corn, tapioca, or sugarcane. PLA is used in FDA approved implants and has undergone extensive biocompatibility testing. PLA is also a biodegradable material meaning that the body can safely reabsorb implants made from PLA. This is an important feature for embeddable photobiomodulation delivery mechanisms, allowing the implant to be absorbed as the body heals and no longer requires the light's effect.

As a polymer, Polylactic Acid (PLA) is transparent when either fully crystalline or fully amorphous. Lactic acid is a chiral molecule. Crystalline PLA is formed by the condensation of lactic acid monomers or lactides that are either the D or L enantiomer rather than a racemic mixture. If starting with a crystalline PLA, exposure of the crystalline PLA at boiling temperatures the crystalline form of the PLA is hydrolyzed. The hydrolyzed polymer chains are shorter and the bulk crystalline form of the polymer becomes a mix of crystalline and amorphous regions. This creates the scattering boundary conditions and the PLA loses its transparency. The amount of hydrolysis is controllable by increasing the exposure time to the boiling temperature water to increase the hydrolysis and decreasing the exposure time to the boiling temperature water to decrease the hydrolysis. By controlling the amount of hydrolysis the transparency of the crystalline form of PLA can be controlled.

By varying the exposure to the boiling temperature water along the length of the scaffold in the manufacturing process, with more exposure at the point or points the light enters the scaffold and less exposure at the farthest point or points from the source, the optical scattering of the scaffold can be engineered to balance the high optical intensity at the point or points where the light enters the scaffold. This balancing allows the optical dose of photobiomodulating light provided to the cells growing on the scaffold to reach a more optimal level where cells near the point or points where light enters the scaffold to be dosed with a similar amount of light to cells growing on the scaffold at the farthest distance from where the light enters the scaffold.

### Example 2: Increased Light Scatter by Creation of Surface Defects

In one embodiment surface defects on the waveguide were generated using sodium hydroxide (NaOH) as an etchant. A 1.75 mm PLA waveguide was obtained from BuMat. This material is sold primarily for 3-D printing and is sold on spools by the manufacturer. If straightening is desired, such can accomplished by holding the PLA in near-boiling water for 10 seconds and then holding it taut while it cooled. Next, the waveguide was heated to 210° C. and melted onto the surface of the glass lens of a 5 mm diameter laser. The lens-waveguide assembly was then removed from the laser by unscrewing the lens from the laser. This lens-waveguide assembly was then placed in a beaker of warm 5M NaOH solution for 10 minutes with continuous magnetic stirring. As a last step, it was rinsed in cold de-ionized water. This scattering-enhanced waveguide was then placed on a microscope and reattached to the 5 mm diameter laser to measure optical scattering at a wavelength of approximately 632 nm.

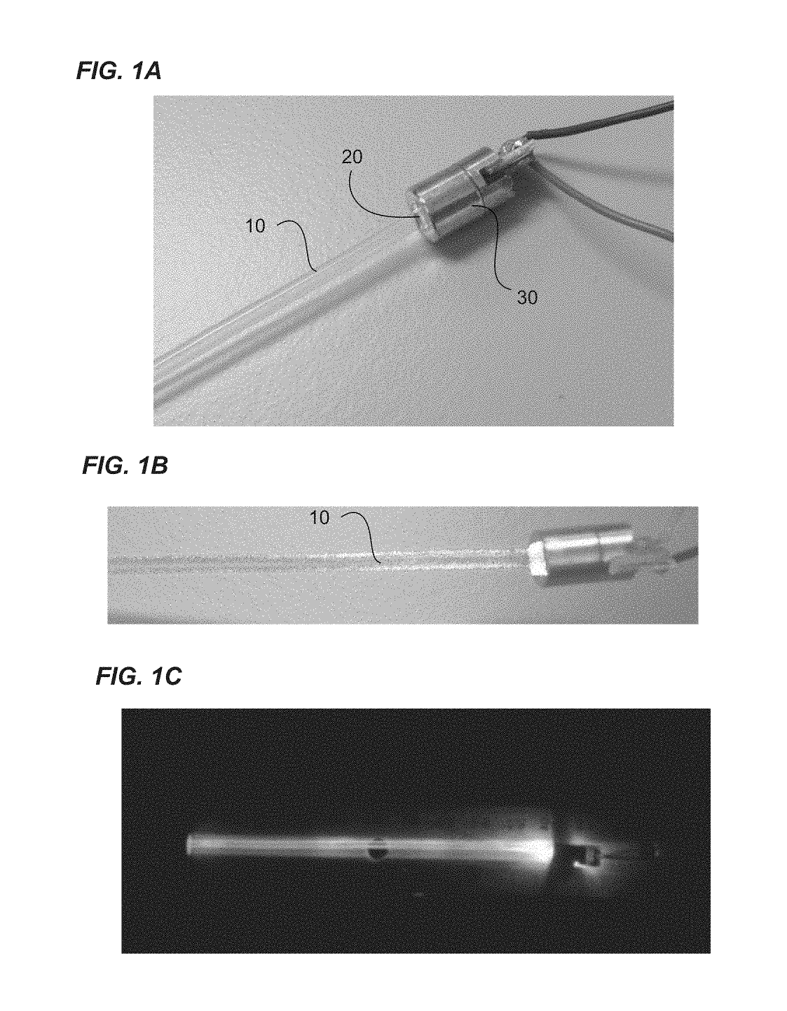

The apparatus depicted in FIG. 1A was built to apply light to the waveguide and to measure scattering. Waveguide fibers 10 were melted to the lens 20 of a minilaser 30 using a hot air gun. The laser-fiber assembly was placed inside of a black box with a charge coupled device (CCD) to take images of the scattered light. The laser was then connected to a 3V DC power source outside the container through a small hole in the container wall. By taking pictures of the illuminated PLA the scattering could be quantitated with a computer program designed to sum the pixels of red light. The power of the light was measured from the end of the fiber and from the surface of the fiber with a Thorlab's PM1002-S120VC power meter.

The NaOH surface etched waveguide had markedly increased optical scattering visible to the naked eye as shown in FIG. 1B. As expected, the scattering appeared limited to the surface. When the experiment was repeated with an etch time of 10 minutes in room temperature NaOH rather than hot NaOH, no change in scattering was observed. This indicates that temperature plays an important role in the formation of surface defects during the etch process or in the rinsing of the PLA after the etch process is complete.

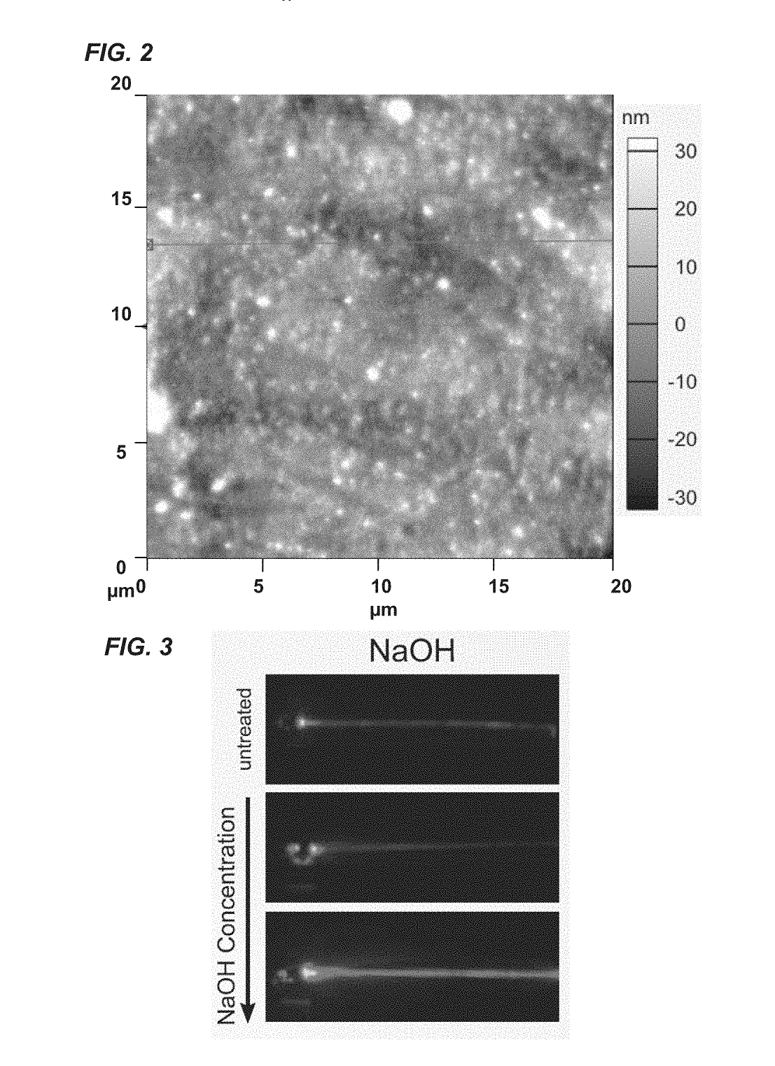

FIG. 2 provides an Atomic Force Microscope (AFM) image taken of the surface of a PLA fiber prior to treatment. FIG. 3 provides a photographic image of the effect of increasing concentration of NaOH on light scattering from the surface of a light wave compared with the untreated PLA waveguide.

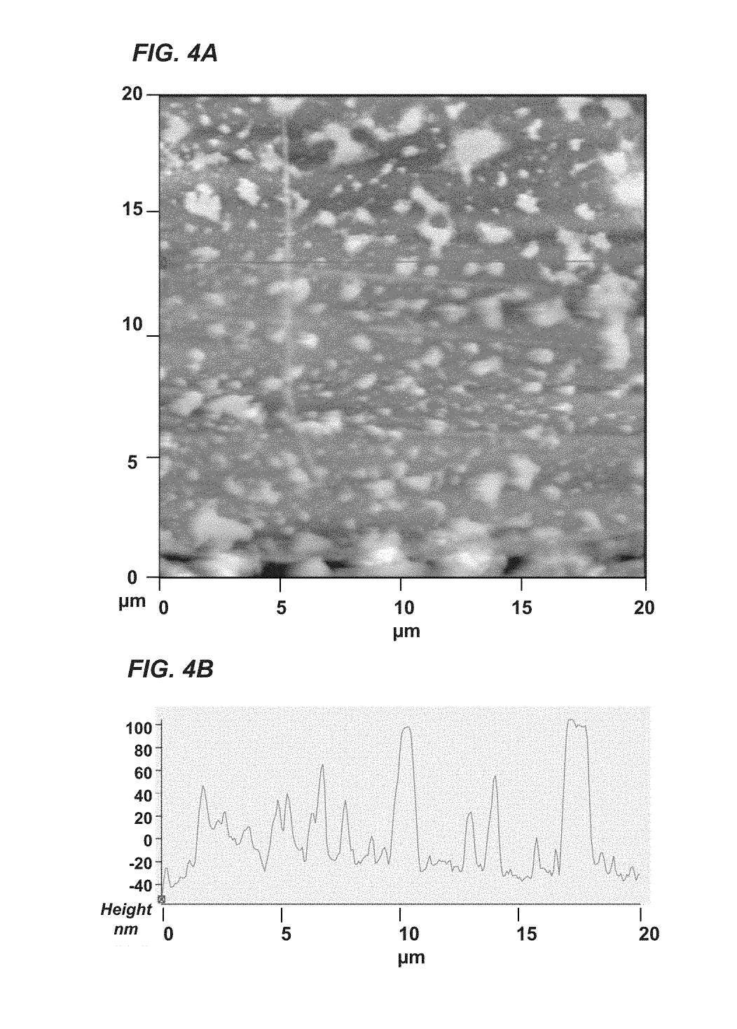

FIG. 4A provides an AFM image of the surface of a PLA fiber treated with 0.5 M NaOH. FIG. 4B is a slice of AFM showing increased roughness (range in Y axis) vs FIG. 2.

### Example 3: Increased Light Scatter by Creation of Sub-Surface Changes

In one embodiment subsurface changes were created in a transparent PLA to increase both optical scattering and optical absorbance. In this method the PLA was again straightened and attached to the glass lens of a 5 mm diameter laser. Next the lens-waveguide assembly was exposed to hot water vapor in a microwave by either wrapping the waveguide assembly in a damp paper towel or by draping the waveguide assembly over a beaker of water and microwaving the assembly for increasing intervals of time at the microwave's highest power. Essentially the microwave heats the damp wrapping and changes the PLA structure to create index boundaries that scatter light. It is believed that the microwave affects the PLA by hydrolysis with water on the surface of the PLA at high temperature. The microwave is not the only way to create this effect as the process essentially requires high temperature PLA and water. The longer high temperature PLA and water are together the more hydrolysis happens, which creates the amorphous boundary layers, which in turn create small regional changes in the refractive index, resulting in increased optical scattering. This effect has also been demonstrated boiling the PLA in water.

The scattering was measured over the range of 30-80 second intervals in 10 second increments. At the end of each increment the waveguide-lens assembly was allowed to cool, reattached to the 5 mm diameter laser, and the amount of optical scattering was measured with a CCD.

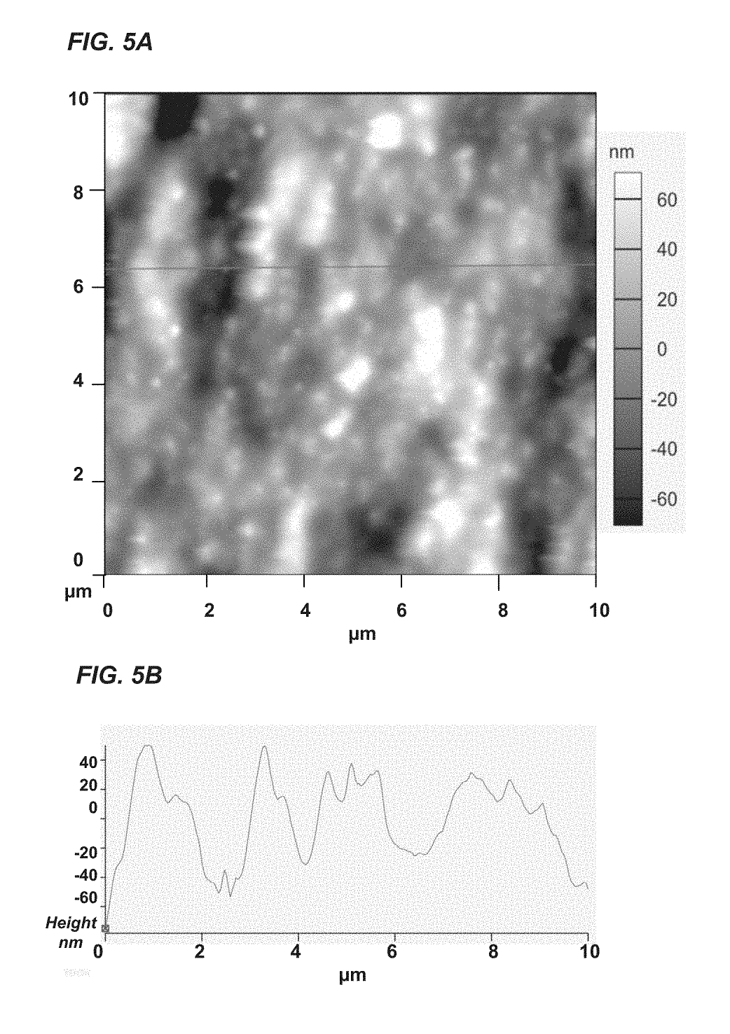

The microwave PLA subsurface scattering experiment also resulted in marked increased in scattering. FIG. 5A provides an AFM image of the surface of a PLA fiber treated by microwave heat in the presence of surface water. FIG. 5B is a slice of AFM showing increased roughness (range in Y axis) vs FIG. 2.

The process appears to be both temperature and water dependent. Heating the PLA to its melting point and then allowing it to cool resulted in no change in scattering while heating it in water vapors either in the microwave or on a hotplate resulted in increased scattering. The treated PLA also had an observably increased rigidity and a higher melting point. Reheating the treated PLA to its melting point reset it to a transparent state.

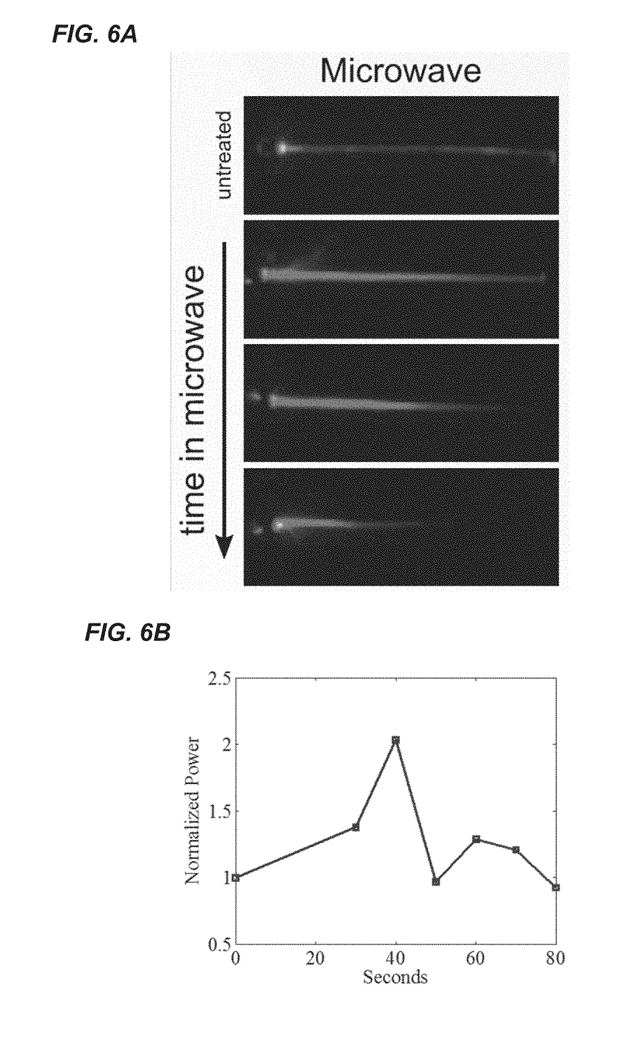

The microwaved PLA's scattering was controllable by increasing the microwave interval. With each 10 second increment of the microwave interval the scattering first increased and then over time absorption dominated as shown in FIG. 6A and FIG. 6B.

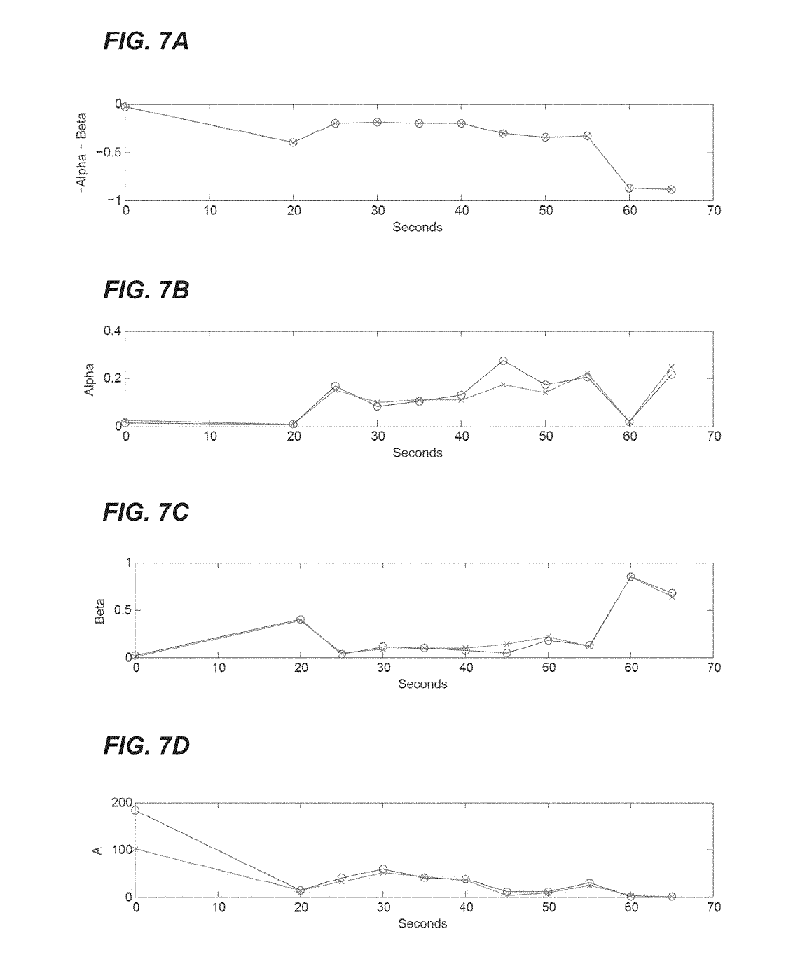

Further analysis of the effects of microwave time and scattering are provided in FIGS. 7A-D. All of the plots are against microwave exposure time in the x axis. Alpha is the exponential factor due to optical scattering and Beta is the exponential decay factor due to absorption. The two lines show two different measurements of the same PLA samples.

FIG. 7A shows that the combination of scattering and absorption increase with microwave exposure time. That is, the more time in the microwave, the less light makes it out the other side of the fiber, some being lost to scattering and some to absorption.

The next two plots break out what was due to scattering and what was due to absorption. FIG. 7B shows scattering increasing with microwave time (except for the 60 s data point, which had too high an absorption to measure the scattering). FIG. 7C shows absorption over microwave time. It stays fairly constant and then jumps up at 60 s. The final plot in FIG. 7D shows optical amplitude. As shown in these experiments, a desired amount of scattering can be engineered by processing the PLA with various levels of heat and water exposure, in this case provided by microwaving.

### Example 4: Optically Transparent Photobiomodulation Scaffolds

In one embodiment, biological scaffolds are provided that utilize transparent materials to allow light to enter and diffuse throughout the scaffold and thereby throughout the cells surrounding and adhered to the scaffold. The application of certain frequencies of the electromagnetic spectrum, specifically optical frequencies in the visible (about 380 to 700 nm) and near-infrared regions (about 700 to 1400 nm) to cells is used to increase cell viability and growth and thereby increase the speed of tissue regeneration. In particular embodiments, a light source provides a coherent, monochromatic light at a selected wavelength selected from a range of wavelengths from about 620 nm to about 760 nm.

The enhanced tissue growth in response to the applied light has been shown to occur at an optimal optical power. Increasing the optical power beyond this point is detrimental to tissue regeneration. Due to high optical attenuation of many tissues, a much higher optical power must be used at the surface of the tissue to allow enough optical power in the depth of the tissue. This prevents the optimal optical power being applied throughout the tissue. The areas of the tissue closer to the optical source will either have too much optical power or the areas of the tissue far from the source will have too little optical power. As a solution to this problem, an optically transparent tissue-embeddable biological scaffold is provided that allows optical power to be more equally applied throughout the tissue, allowing for more optimal photobiomodulation.

The materials used to create the scaffold must be both biocompatible and optically transparent or the scaffold must contain at least some transparent elements to guide the light throughout the scaffold. Any biocompatible and transparent material can be used that has sufficient structural rigidity to be formed into a scaffold. Three examples include, polylactic acid (PLA), silk fibroin and polyethylene glycol. PLA is commonly used for medical implants, has been rigorously tested for biocompatibility, and is available in transparent form. Silk fibroin, extracted from silk-worm silk, is both biocompatible and optically transparent. Silk fibroin can be mold-formed or spun to form a scaffold. Polyethylene glycol (PEG) is commonly used in tissue scaffolds, can be formulated to be transparent, and has been shown to be biocompatible.

In one embodiment, in order to distribute light evenly throughout the scaffold, the material is treated to scatter less light at the point where the light enters the scaffold and more light at the periphery of the scaffold to make up for the light leaving the scaffold between the source and the periphery. Several material processes have been tested to create this scattering gradient. In one process the scaffold material is embedded with scattering elements, nano particles or bubbles, such that the concentration of the scattering elements is greater where the light enters the scaffold and less at the periphery of the scaffold.

Another process involves creating roughness on the surface of the material through the application of an etchant to the surface of the material. One non-limiting example of an etchant applicable to PLA is a solution of sodium hydroxide (NaOH) applied to the surface in varying concentrations to allow the surface scattering of the PLA to be controlled, creating a scattering gradient with less scattering where the concentration of NaOH is less and more scattering where the concentration of NaOH is increased.

A further process is to create scattering within the material through the generation of molecular optical boundary surfaces. These boundary surfaces exist when regions of the material contain quickly varying optical indices of refraction. As an example, for PLA, these boundaries can be generated by heating a wetted surface of the PLA such as has been demonstrated here by first wrapping the scaffold in a water-moistened sponge, paper-towel, or cloth and then microwaving the wrapped scaffold, As disclosed herein, with longer exposure to the microwaves, the scattering of the PLA increases.

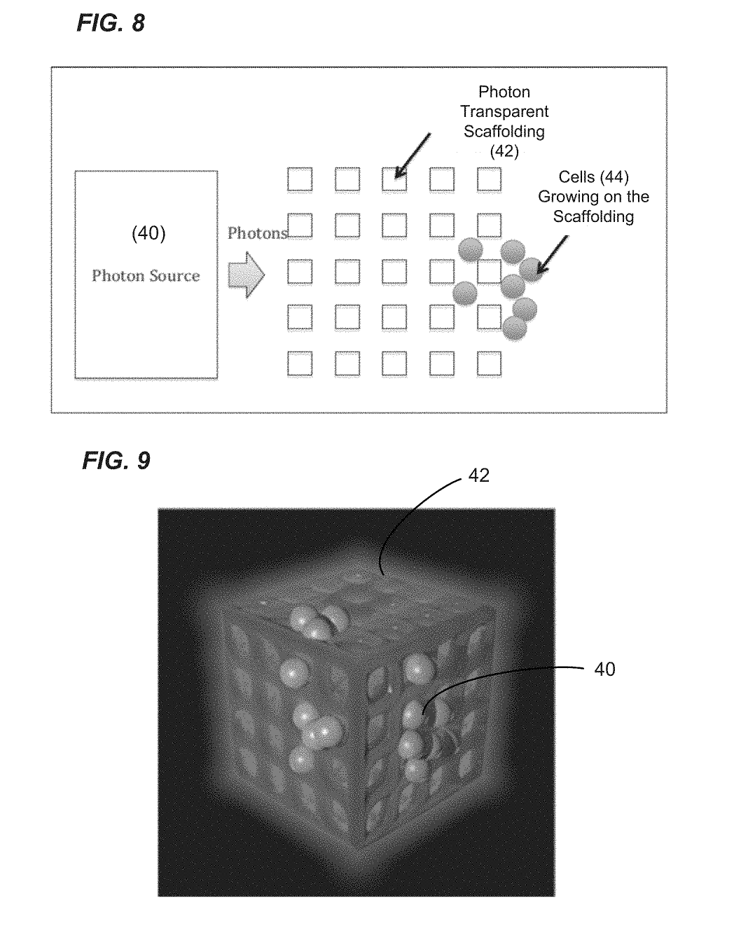

The photons of light from the source are guided by a transparent scaffold structure to the cells of the tissue growing within and around the scaffold to activate biological processes within the cells to increase the growth rate of the tissue or selectively increase the growth rate of specific cells within the tissue. The scaffold may be used in vivo when the scaffold is implanted during surgery inside of a patient to deliver light to specific organs or tissues, or within a wound to deliver light to the wound as the wound heals. The scaffold may also be used in vitro when placed inside of a bioreactor to generate cells and tissues outside of the body. FIG. 8 depicts a cartoon of an embodiment showing one configuration of a photon transparent scaffolding (42) with cells (44) growing on the scaffold and light supplied by photon source (40). The figure is not shown to size and the voids in the scaffold may be larger or smaller than depicted and have different configurations.

FIG. 9 depicts a 3D rendering of an embodiment showing one configuration of a photon transparent scaffolding (42) illuminated with light and with figurative cells (44) growing on the scaffold. The figure is not shown to size and the voids in the scaffold may be larger or smaller than depicted and have different configurations. As shown, the scaffold can have a frame formed by vertical and horizontal elongated support members that overlap with one another and have openings therebetween. The scaffold is in the shape of a cube, though other geometries can be utilized.

Light for photobiomodulation can be coupled into the scaffold from either from a light emitting diode (LED) or LASER source. The source can be either internal in which case the source is placed inside the scaffold or external in which case the light from the source is directed to the scaffold from outside of the body. In the case of an external source, light can be guided to the scaffold from an optical waveguide made from a portion of the scaffold itself, or light can be guided to the scaffold through a secondary guiding optical element. This guiding optical element can be a medical optical fiber such as those commonly used in other optical medical applications or it can be a variant of a catheter.

Dual-Use Catheters:

Catheters are commonly placed in wounds to allow drainage of the wound as the wound heals. A catheter made of a biocompatible and optically transparent material can be used to both drain the wound and supply the wound and scaffold implant with photobiomodulating light. In one embodiment, two cladding layers of a lower optical index of refraction material are placed on either side of the core catheter material to enhance the optical carrying ability of the catheter. This allows the catheter to carry light inside its surface in a similar manner to an optical fiber.

To continue to function as a liquid carrying catheter, the catheter is coupled on the outside and inside of the body to both a liquid and optical path. The optical path on the outside of the body connects to the photobiomodulation optical source and on the inside of the body connects to the implant. The liquid path connects to the wound on the inside of the body and to a waste container on the outside of the body. The point at which the optical and liquid paths meet both inside and outside of the body are designed with thin smooth surfaces to guide the light to and from the surface of the catheter with minimal optical scattering and losses.

The catheter continues to function in its primary capacity to transport liquid into and out of the wound. After the wound heals, the catheter is pulled to detach it from the implant and removed from the body.

FIG. 11a depicts one embodiment of a light attachment to a scaffold where light coupling conduit (46) or dual use light conduit catheter combination (76) extends outward from photon transparent scaffolding (42) and conveys light from a light source to the scaffold placed within the body. As depicted, the dual use light conduit catheter combination extends at a right angle to the scaffold such that the light conduit and catheter can be coupled orthogonally to an implant site. In one embodiment, the optical coupling (48) between the scaffold and the light conduit is thin and branching to allow flexibility in scaffold placement. In one embodiment, the light coupling conduit (46) is attached to the scaffold through a coupling that essentially provides a connection between an optical conduit running from the light source to the light coupling conduit of the scaffold. In one embodiment the coupling is a pressed fitted attachment such that the light conduit can be pulled free of attachment to the scaffold and the scaffold left in the body, while removing the light conduit that attached to the light source.

FIG. 11b depicts a close-up view of a scaffold implant catheter attachment tube. Light coupling conduit (46) or conduit catheter combination (76) extends outward from the scaffold and conveys light from a light source to the light distribution mesh that forms the scaffold (42). The optical coupling attachment (48) is formed as a plurality of relatively thin legs that allow flexibility in scaffold placement to the wound and the curved surfaces couple light to the light distribution mesh of the scaffold. Essentially, light coupling conduit (46) splits to form the plurality of legs of the optical coupling attachment (48) that forms the optical connection to the scaffold. The legs can be curved and extend outward from the conduit (46). Each leg can further connect with the ring resonators, such as at a point where four ring resonators come together. The legs can carry light to the ring resonators and/or direct fluid from the conduit (46).

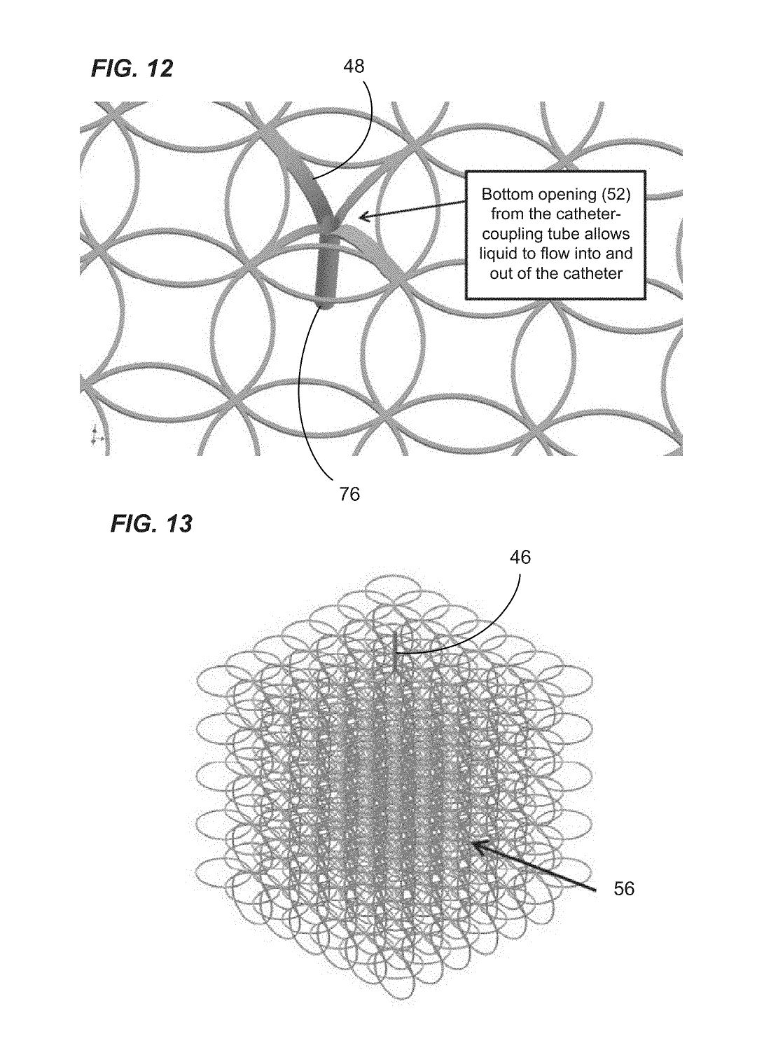

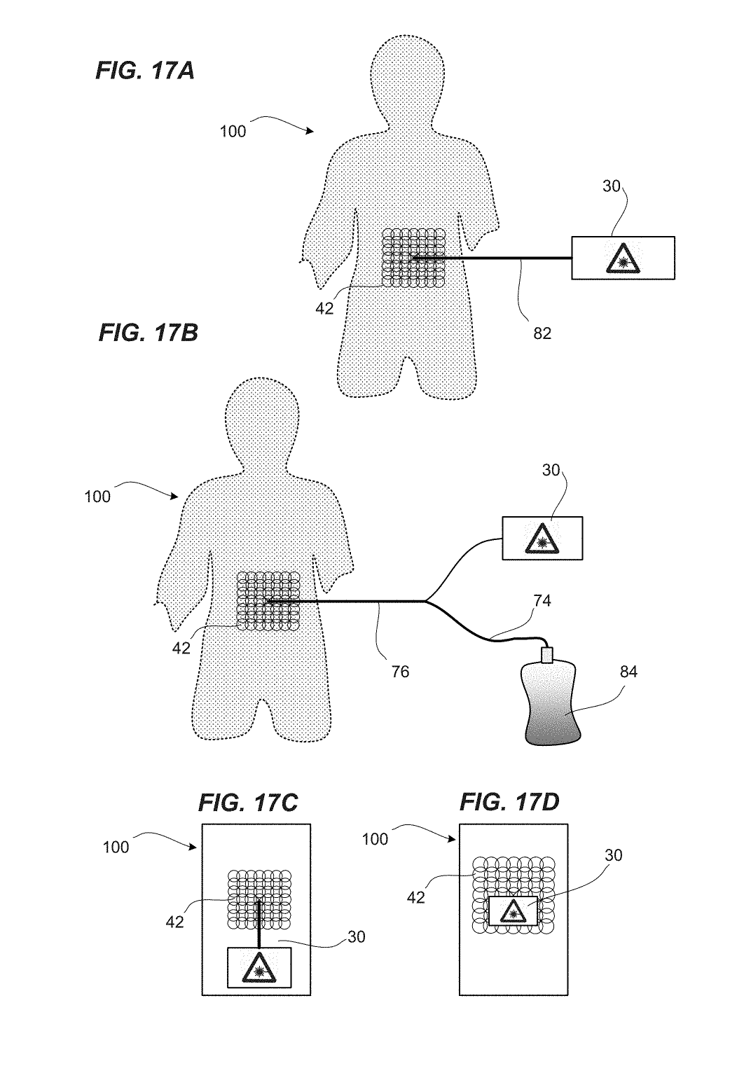

FIG. 12 depicts an underside view and shows bottom opening (52) of the catheter-coupling tube that allows liquid to flow into and out of the scaffold (and ultimately out of the body) through the central conduit of the dual use light conduit catheter combination as shown in more detail in FIG. 16A and FIG. 17B.

FIG. 13 depicts a three dimensional embodiment (56) of a mesh scaffold implant formed by a plurality of ring resonators. Light coupling conduit (46) or, if desired, a dual use light conduit catheter combination, extends outward from the scaffold and conveys light from a light source to the 3D light distribution mesh that forms the scaffold. The figure depicts the light coupling conduit on an outer plane of the implant but alternative include situating the light coupling conduit at a central point in the 3 dimensional (3D) implant. The scaffold can be formed by a number of layers of ring resonators, each layer connected in horizontally or vertically.

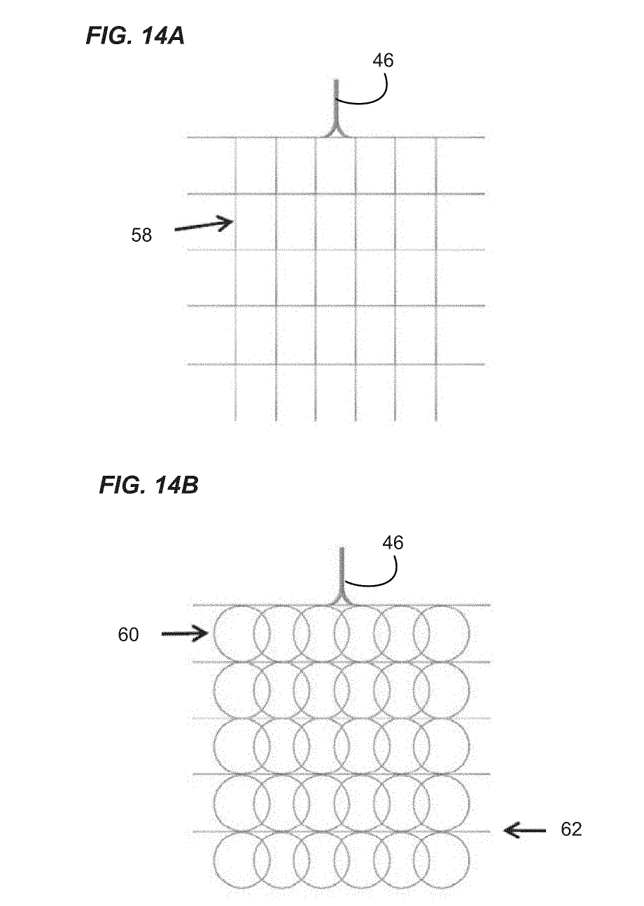

In FIG. 14A a side schematic of one embodiment is provided wherein each set of vertical rings (58) couples sets of 2D horizontal ring arrays to form a three dimensional structure. In the front schematic of FIG. 14B, curved vertical rings (60) connect layers of curved horizontal rings (62). In certain embodiments, most or all of the materials forming the scaffold are transparent and are interconnected thus forming a 3D waveguide mesh that conveys inputted light throughout the mesh. Preferably the interconnected rings of the scaffold are dimensioned to form a complex of voids through which cells can move through the implant as well has fluid that conveys nutrients into the scaffold and allows waste to be removed. Thus, in certain embodiments, the scaffold provides interconnected voids for cell passage dimensioned to allow movement of cells having an average diameter of 10-30 μm (microns) through the scaffold.

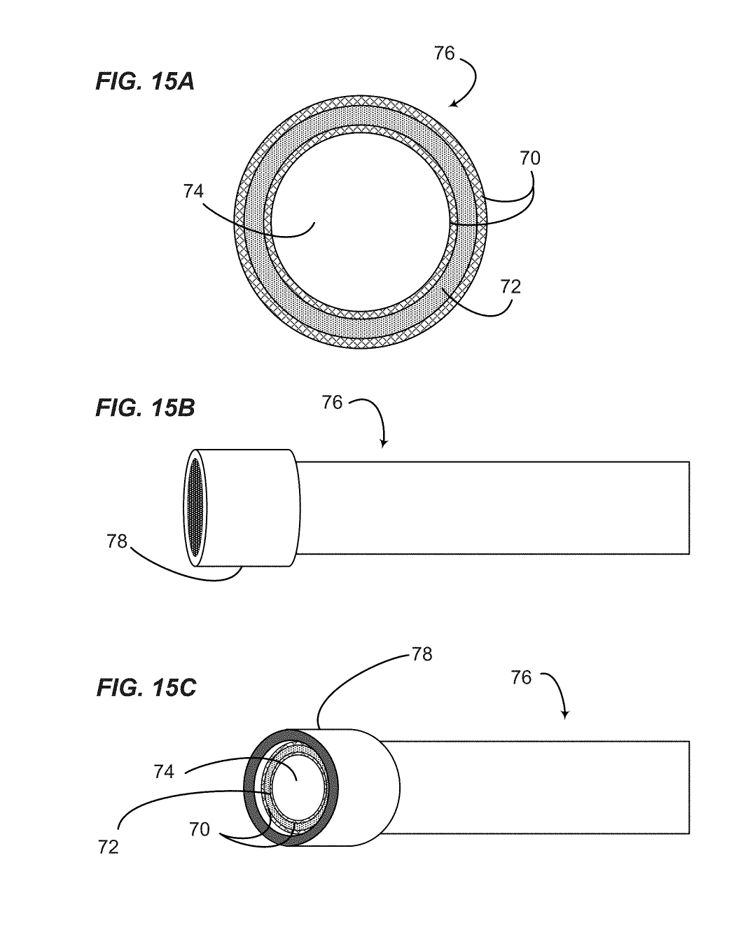

FIG. 15A provides a cross section of one embodiment of a dual use light conduit catheter combination (76). An outer light guide is formed by the combination of cladding layers (70) having a lower index of refraction than the light transmitting core layer (72), which has a higher index of refraction than the cladding. Thus, there is an inner layer (70) and an outer layer (70), the two layers being concentric, with (72) therebetween. Fluid flows through central conduit (74). In one embodiment depicted in FIG. 15B, a connecting flange (78) around an end of dual use light conduit catheter combination (76) provides a mechanical connection to the scaffold by applied radial tension. FIG. 15C shows an end view of dual use conduit (76) within flange (78). By virtue of the fitted flange attachment, the dual use light conduit catheter combination can be removably attached and pulled free of the scaffold thus allowing removal of the dual use light conduit catheter combination while leaving the scaffold in place after the photomodulation is completed. The flange (78) can have a larger diameter than the conduit (76), so that the flange (78) can couple with the scaffold and allow light to travel to the conduit (76) uninterrupted by the flange (78).

FIG. 16A provides an embodiment of a splitter for a dual use light conduit catheter combination (76) where the central conduit (74) leaves the combined light conduit (76). In this embodiment a small angle joint (80) is provided to prevent optical scattering from the optical path (81) as it enters the combined light conduit catheter (76).

FIG. 17A depicts placement of scaffold (42) in a patient (100), although not drawn to scale. Optical fiber (82) transports light from a source (30) outside of the body of the patient (100) to the scaffold inside the body. In one embodiment optical fiber (82) connects to optical coupling conduit (46) through flange (78) as depicted in FIG. 15B. As depicted in this and other figures, a laser symbol is placed on the source but this symbol is meant to depict any light source including lasers and light emitting diodes. In the embodiment depicted in FIG. 17B, a dual use light conduit catheter is provided. Scaffold (42) is located inside a patient (100). Dual use light conduit catheter (76) transports light from outside of the body of the patient (100) to the scaffold inside the body, which fluid conduit (74) provides a pathway for transporting fluids into the scaffold or draining fluids out of the scaffold implant site via bag (84). In alternative embodiments such as depicted in FIG. 17C, the light source (30) may be inside the body (100) and optically coupled to the scaffold (42) or may be located on or in the scaffold as depicted in FIG. 17D. In such cases, the light source may be optionally coupled to a miniature implanted battery that is designed to have sufficient power to operate the light source throughout the desired healing process. In certain embodiments, the battery may be recharged from outside the body by radio frequency charging. Once healing is sufficiently complete, the light source and battery may be removed as conveniently, particularly where placed under the skin.

As an alternative to direct in vivo implantation, the photobiomodulation scaffolds disclosed herein can be seeded with cells ex vivo in order to jump start the growth of cells on the scaffold. For medical implants the tissue may be grown in vitro in a bioreactor and then implanted in the patient, the tissue may be grown in vivo in a host animal and then moved to the patient, or the tissue may be grown in vivo within the patient.

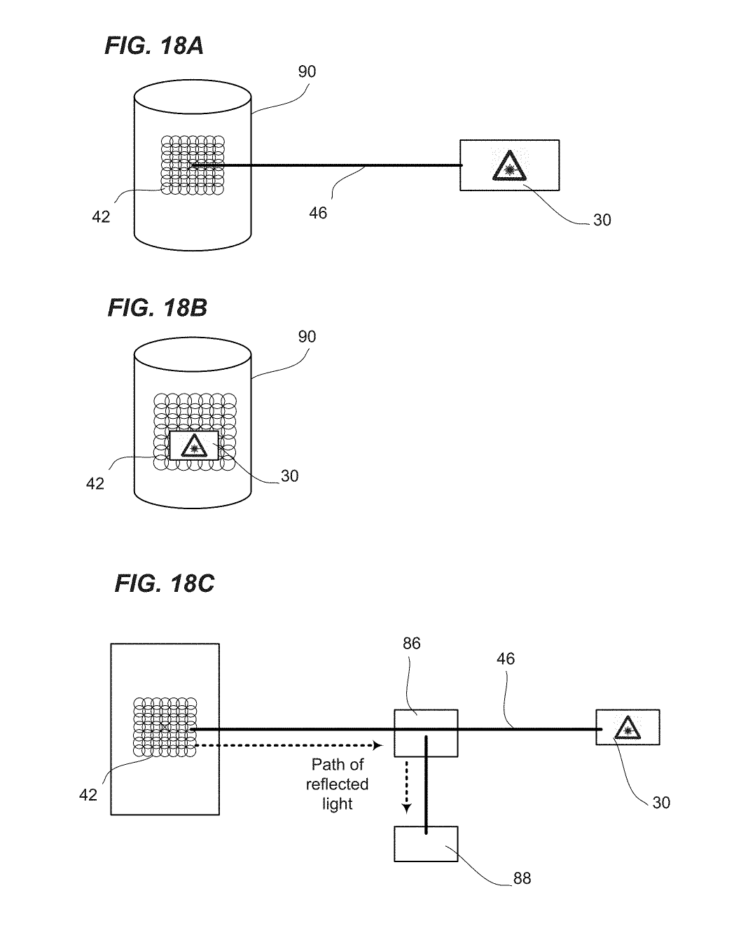

As depicted in FIG. 18A, the light conductive photobiomodulation scaffold (42) is placed in a bioreactor (90). The light source (30) can be external to the bioreactor as in FIG. 18A or can be located within the bioreactor as depicted in FIG. 18B. Cells for seeding on the scaffold will preferably be autologous, such as autologous mesenchymal stem cells. Growth factors provided in the feed media are added to encourage differentiation of the stem cells to the lineage desired for the site of the implant.

Whether utilized ex vivo or in vivo or combinations thereof, in certain embodiments the scaffold is provided with bioinstructive coatings including one or more components of the extracellular matrix (ECM) including collagen, ligands, growth factors, adhesion peptides, cytokines, and gene delivery vectors thereof. The scaffold may in certain embodiments be provided with inflammatory inhibitors, heparin, and/or antibiotics to reduce the possibility of inflammation, clotting and infection after surgery for implantation.

In other embodiments, the scaffold is tailored to the mechanical properties inherent to the implantation site. In certain embodiments, the stiffness of the scaffold is adapted to the mechanical properties desired for the cells that are intended to functionally colonize the scaffold at its site of implantation. For example, mesenchymal stem cells will respond to a stiff substrate by differentiating down an osteogenic pathways while adaptation of the scaffold to the elasticity of muscle will encourage differentiation down a myogenic lineage pathway. In certain embodiments were the scaffold is intended to mature down an osteogenic pathway such as for bone grafts and spinal fusions, load stress is applied to the scaffold during ex vivo growth in the bioreactor. Likewise, where utilizing neural stem cells, neuron differentiation is encouraged by use of a soft scaffold that mimics normal brain tissue, while differentiation into neuron-supporting glial cells is encouraged with a more rigid scaffold.

Sensing with Light Reflected Back from the Scaffold:

In certain embodiments, light reflected back out of the scaffold, whether the scaffold is placed in vivo or ex vivo, is measured and analyzed to monitor the progress of tissue growth in the scaffold. Changes in both the amplitude and the frequency components of reflected light are indicative of tissue growth progress. As depicted in FIG. 18C, an optical splitter (86) placed in the optical path (46) between the light source (30) and the scaffold is used to collect light reflecting back from the scaffold, sending the reflected light to an optical sensor (88) such as a power meter or optical spectrometer.

### Example 5: Implant Structures

Bone Implants:

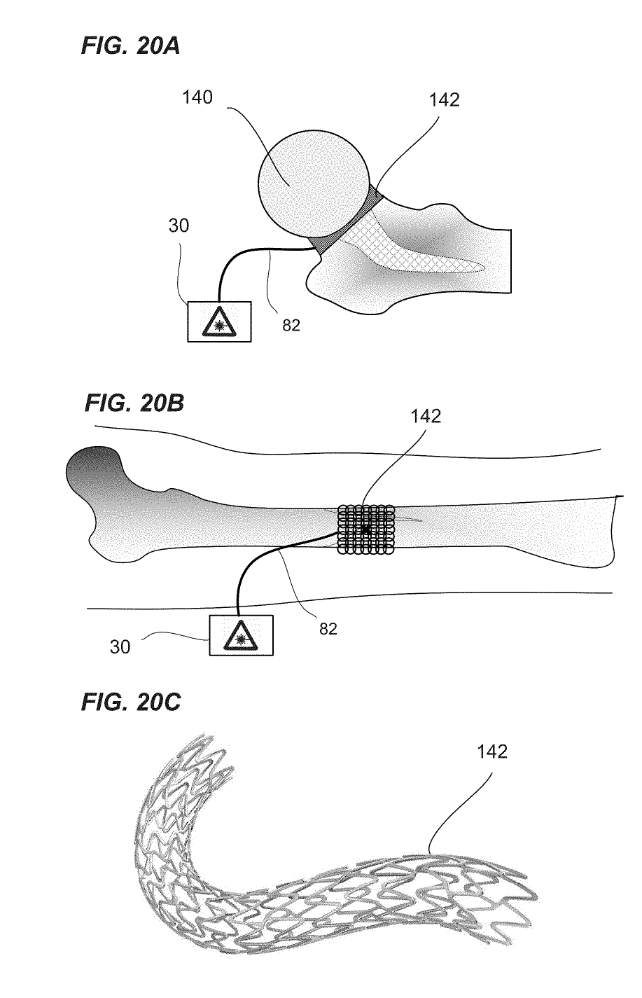

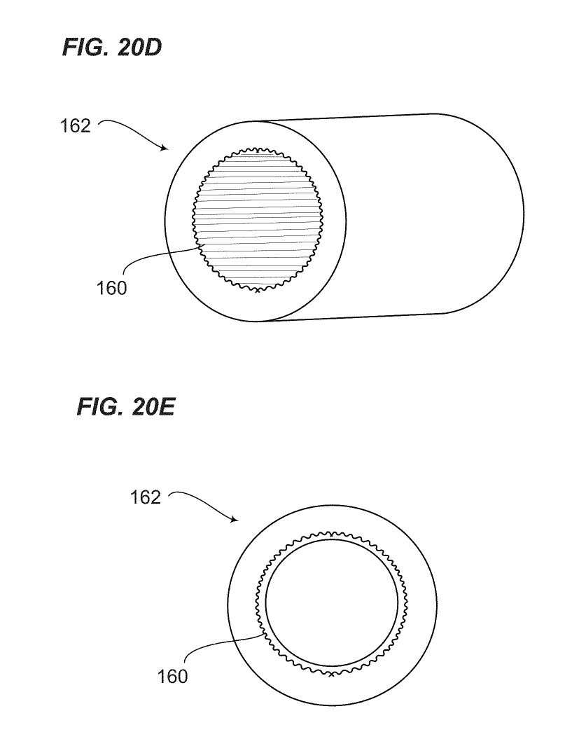

In certain embodiments, a portion of a bony structure is replaced with the photobiomodulation tissue scaffold. The seeded tissue scaffold may be incubated outside of the patient, in a bioreactor or in an animal host, or may be directly implanted in the patient. Photobiomodulating light is delivered to the tissue as it incubates in the bioreactor or in the animal host. Photobiomodulating light may also be delivered to the implant after it is placed in the patient. In the depicted embodiment of FIG. 20A, a portion of the artificial hip (140) that touches the remaining thighbone is covered in the photobiomodulating tissue scaffold (142). Scaffold (142) can be coupled to the target (bone, tissue, etc.) as desired by the surgical team including one or more of sewing, gluing, stapling, screwing and wrapping. After being implanted, photobiomodulating light from a source (30) outside of the body is dosed into the scaffold decreasing inflammation and increasing the rate of bone tissue formation. FIG. 20B depicts an embodiment where a photobiomodulating tissue scaffold (142) is used to aid in non-union fracture repair or even to form a portion of damaged or missing bone. After being implanted, photobiomodulating light is transmitted through optical guide (82) from a source (30) outside of the body is dosed into the scaffold decreasing inflammation and increasing the rate of bone tissue formation. Alternatively, the source and associated battery may be implanted under the skin. After healing is sufficient, the source and battery are removed via a small incision and the scaffold is left in place to biodegrade.

Stents:

One embodiment provides stents formed from a biodegradable transparent material. After implantation, the stent is dosed with photobiomodulating light. The photobiomodulating stent may optionally include an internal light source powered through induction for later optical dosing. This decreases inflammation and increases the speed of endothelial cells growing at the site decreasing the potential for scar tissue formation and restenosis. FIG. 20C depicts a SYNERGY™ biosorbable polymer stent from Boston Scientific merely for illustrative purposes. The actual conformation of the stent is not important as long as it is formed as a transparent interconnected waveguide mesh scaffold that delivers photobiomodulating light throughout the stent as well as mechanical structure to the stenosis region of the artery.

Spinal Cord Implants:

One embodiment provides for repair of a damaged portion of the spinal cord by implanting photobiomodulating tissue scaffold that directs light into the growing nerve tissue and guides the nerves along the damaged portions of the spinal cord to reconnect nerves across the missing segment. As depicted in FIG. 20D, the waveguide (162) may be designed to include grooves (160) in the inner surface of the wave guide to direct nerve growth.

Brain Implants:

A damaged portion of brain tissue may be replaced with a photobiomodulating implant or tissue. Alternatively, a stimulating or sensing nerve interfacing implant is supplemented with the photobiomodulating implant to encourage more rapid nerve regeneration.

Organ Tissues:

Organ tissues are grown on a scaffold that is with varying stem cell types. Such cell types may be strategically placed on the implant with 3D printing of the cells. The stem cells are placed within the tissue to eventually form a replacement organ. The tissue scaffold is printed with embedded transparent photobiomodulating optical waveguides. These waveguides delivers photobiomodulating light into the growing organ, either in vitro or in vivo. The photobiomodulating light reduces inflammation and increases the rate of tissue formation.

Vascular Repair Implants:

One embodiment provides photobiomodulating tissue scaffolds that are adapted and dimensioned to repair vascular defects including cardiovascular defects. Non-limiting examples include use of the photobiomodulating tissue scaffolds as carotid patches, aortic grafts, aortic and other aneurism patches, vascular grafts, congenital defect reconstruction grafts, dialysis access grafts, and peripheral vasculature patches including for repair of carotid, renal, iliac, femoral, and tibial blood vessels. In certain of these embodiments, photobiomodulating tissue scaffolds are provided that are manufactured with smaller voids such that the scaffold operates as do existing knitted or woven patches that that prevent loss of red blood cells through the patch.

Hernia Repair Implants:

Biocompatible mesh has been used for hernia repair for over 50 years and is considered superior to suture repair. Despite the vast selection of brands available, nearly all meshes continue to use one of three basic materials—Polypropylene, Polyester and ePTFE. None of these synthetic materials are without disadvantages and more physiologically based implants have been recently introduced including an acellular collagen matrix or porcine small intestine submucosa. However, while these biological matrices allow soft tissue to infiltrate the mesh and integration into the body by remodeling, this process also leads to rapid reductions in mechanical strength that has restricted their use to infected environments. It is clear that the ideal mesh has yet to be found. One embodiment provided herein is a photobiomodulating tissue scaffold that encourages cell recruitment and growth on the scaffold while retaining structural integrity until the tissue is sufficiently rebuild. Thus, in one embodiment a photobiomodulating tissue scaffold is adapted and dimensioned as a hernia repair mesh patch that is connected via an optical conduit to a light source. The light source and power supply can be mounted under the skin or placed in an adhesive patch on the outside of the body. When repair is sufficiently complete, the optical conduit, as well as the light source and power supply if need be, is pulled from the tissue scaffold, which is left in place.

Prosthetic Implants:

In one embodiment, 3D printed anatomically correct transparent PLA tissue scaffolds are provided for implantation under the skin to repair cartilaginous defects such as with congenital defects and with reconstructive surgery for injury or disease, particularly to the ear or nose.

Food Protein:

In one embodiment, cells are seeded onto a photobiomodulating scaffold. The scaffold provides structure and light to the forming tissue. The structure allows the tissue to form providing texture to the resulting artificial protein. The photobiomodulating light increases yield by delivering exact optical doses into the growing tissue.

It is noted that the description uses several geometric or relational terms, such as circular, ring, orthogonal, square, cube, and concentric. In addition, the description uses several directional or positioning terms and the like, such as top, bottom, left, right, up, down, inner, and outer. Those terms are merely for convenience to facilitate the description based on the embodiments shown in the figures. Those terms are not intended to limit the invention. Thus, it should be recognized that the invention can be described in other ways without those geometric, relational, directional or positioning terms. In addition, the geometric or relational terms may not be exact. For instance, the conduit (46) need not be exactly perpendicular to the scaffold (42), but still be considered to be substantially perpendicular because of, for example, roughness of surfaces, tolerances allowed in manufacturing, etc. And, other suitable geometries and relationships can be provided without departing from the spirit and scope of the invention. For instance, the conduit (46) need not be perpendicular to the scaffold (42).

All publications, patents and patent applications cited herein are hereby incorporated by reference as if set forth in their entirety herein. While this invention has been described with reference to illustrative embodiments, this description is not intended to be construed in a limiting sense. Various modifications and combinations of illustrative embodiments, as well as other embodiments of the invention, will be apparent to persons skilled in the art upon reference to the description. It is therefore intended that the appended claims encompass such modifications and enhancements.

We claim:

1. A device for tissue repair comprising:
a tissue scaffold comprising of a plurality of interconnected photon waveguides, the waveguides adapted to convey cell stimulatory photons and to release the cell stimulatory photons from the waveguides by optical scattering, and
an optical connector attached to the tissue scaffold, wherein the optical connector is adapted to connect to a source of cell stimulatory photons.

2. The device of claim 1, wherein the waveguides are biodegradable.

3. The device of claim 2, wherein the biodegradable waveguides are composed of a transparent material selected from the group consisting of: transparent polylactide (PLA), silk fibroin, and polyethylene glycol (PEG).

4. The device of claim 1, wherein the tissue scaffold is formed as a plurality of interconnecting ring resonators.

5. The device of claim 4, wherein the tissue scaffold is formed as a three dimensional mesh of interconnecting ring resonators.

6. The device of claim 3, wherein the waveguides are treated to increase optical scattering.

7. The device of claim 1, wherein the waveguides are composed of PLA or silk fibroin and are treated by surface etching to increase optical scattering.

8. The device of claim 1, wherein the waveguides are composed of PLA or silk fibroin and are heat treated to generate amorphous boundary layers that result in increased optical scattering.

9. The device of claim 1, wherein the tissue scaffold is an expandable stent.

10. The device of claim 1, wherein the tissue scaffold is a bone repair scaffold.

11. The device of claim 1, wherein the tissue scaffold is a muscle repair scaffold.

12. The device of claim 1, wherein the tissue scaffold is a vascular tissue repair scaffold.

13. The device of claim 1, wherein the tissue scaffold is a nervous tissue repair scaffold.

14. The device of claim 1, wherein the tissue scaffold is a 3D printed anatomically correct ear or nose prosthesis.

15. The device of claim 1, wherein the tissue scaffold is a hernia repair scaffold.

16. The device of claim 1, wherein the optical connector is formed to include a central fluid conduit that provides fluid flow into and away from the tissue scaffold.

17. The device of claim 1 further comprising an optical conduit that is adapted to connect the optical connector to a laser or light emitting diode as a source of cell stimulatory photons elaborated by a laser or light emitting diode.

18. The device of claim 17 further comprising a laser or light emitting diode that emits cell stimulatory photons in one or more wavelengths in a range of wavelengths from 620 nm to 760 nm.

19. A method of making a cell seeded tissue scaffold comprising:
providing a tissue scaffold into a sterile in vitro cell growth chamber, wherein the tissue scaffold comprises a plurality of interconnected photon waveguides, the waveguides adapted to convey cell stimulatory photons and to release the cell stimulatory photons from the waveguides by optical scattering;
connecting the tissue scaffold to a source of cell stimulatory photons;
seeding the tissue scaffold with a plurality of cells in a growth medium; and
incubating the tissue scaffold under conditions and for a time sufficient for the cells to colonize the scaffold.

20. The method of claim 19, wherein the plurality of interconnected photon waveguides

21. The method of claim 19, wherein the waveguides are biodegradable.

22. The method of claim 21, wherein the biodegradable waveguides are composed of a transparent material selected from the group consisting of: transparent polylactide (PLA), silk fibroin, and polyethylene glycol (PEG).

23. The method of claim 19, wherein the tissue scaffold is formed as a plurality of interconnecting ring resonators.

24. The method of claim 19, wherein the tissue scaffold is formed as a three dimensional mesh of interconnecting ring resonators.

25. The method of claim 19, wherein the waveguides are treated to increase optical scattering.

26. The method of claim 19, wherein the waveguides are composed of PLA or silk fibroin and are treated by surface etching to increase optical scattering.

27. The method of claim 19, wherein the waveguides are composed of PLA or silk fibroin and are heat treated to generate amorphous boundary layers that result in increased optical scattering.

28. A three-dimensional biocompatible tissue scaffold comprising a biocompatible transparent material that conducts photons provided from a photon source and releases the photons substantially evenly from the transparent material forming the scaffold, wherein the scaffold is formed as an interconnecting array of ring resonators.

29. The three dimensional biocompatible tissue scaffold of claim 28, wherein the interconnecting array of ring resonators comprises a plurality of interconnected voids dimensioned to allow movement of cells having an average diameter of 10-30 μm (microns) through the scaffold.
